(19) United States
(12) Plant Patent Application Publication
(10) Pub. No.: US 2016/0242336 P1
(43) Pub. Date: Aug. 18, 2016

(54) Grapevine named 'ROYAL CABERNET'

(50) ;

(71) Applicants: Università degli Studi di Udine, Udine UD (IT); Istituto di Genomica Applicata, Udine UD (IT)

(72) Inventors: Simone Diego CASTELLARIN, Udine (IT); Guido CIPRIANI, Faedis (IT); Gabriele DI GASPERO, Cividale del Friuli UD (IT); Michele MORGANTE, Tricesimo UD (IT); Enrico PETERLUNGER, Codroipo UD (IT); Raffaele TESTOLIN, Udine (IT)

(73) Assignees: Università degli Studi di Udine, Udine UD (IT); Istituto di Genomica Applicata, Udine UD (IT)

(21) Appl. No.: 14/545,113

(22) Filed: Mar. 27, 2015

(30) Foreign Application Priority Data
Feb. 13, 2015 (QZ) 2015/0362

(51) Int. Cl. A01H 5/00

(52) U.S. Cl. PLT/205

(57) ABSTRACT

A new and distinct variety of grapevine, primarily suited to the growing conditions of the temperate regions, and characterized by its medium vigor; semi-erect growth habit; medium, pentagonal to circular shaped leaves; short-medium, dense, cylindrical berry clusters; dark blue berries with soft to slightly firm berry flesh, herbaceous taste, and no flesh coloration; and resistance to winter temperatures (to −24° C.), resistance to downy mildew, and tolerance to powdery mildew.

### LATIN NAME OF THE GENUS AND SPECIES OF THE PLANT CLAIMED

Vitis×vinifera (hybrid)

### VARIETY NAME

‘ROYAL CABERNET’

### BACKGROUND OF THE INVENTION

The present invention relates to a new and distinct summer/fall bearing grapevine variety, botanically known as Vitis×vinifera, and hereinafter referred to by the name ‘ROYAL CABERNET’.

The new grapevine ‘ROYAL CABERNET’ is a product of a controlled breeding program conducted by the inventors in Udine, Italy. The objective of the breeding program was to develop a new grapevine variety particularly characterized by resistance to cold (<−20° C.), resistance to downy mildew (Plasmopara viticola), and tolerance to powdery mildew (Uncinula necator).

The new grapevine ‘ROYAL CABERNET’ originated from a cross made by the inventors in 2002 in Udine, Italy. The female or seed parent is the grapevine variety, Vitis vinifera cv. ‘Cabernet Sauvignon’ (unpatented), and the male or pollen parent is the grapevine variety, Vitis cv. ‘20/3’ (Bianca x SK77-4/5) (unpatented).

The new grapevine ‘ROYAL CABERNET’ was discovered and selected by the inventor as a single flowering plant within the progeny of the stated cross in a controlled environment in 2002 in Udine, Italy. Asexual reproduction of the new grapevine variety by grafting was first performed in February 2004 in Rauscedo, Friuli Venezia Giulia region, Italy, and has demonstrated that the combination of characteristics as herein disclosed for the new cultivar are firmly fixed and retained through successive generations of asexual reproduction. The new cultivar reproduces true to type.

### SUMMARY OF THE INVENTION

‘ROYAL CABERNET is primarily adapted to the climate and growing conditions of the temperate regions with average yearly temperature about 13° C., minimum winter temperature about −20° C., annual rainfall around 700-1500 mm of rain (e.g. North-Eastern Italy, Friuli). This region provides the necessary year-round temperatures required for it to produce and maintain a strong vigorous plant with consistent fruit production from April through November on primocanes and in the ensuing year from April through November on the floricanes.

The following traits have been repeatedly observed and are determined to be unique characteristics of ‘ROYAL CABERNET’, which in combination distinguish this grapevine plant as a new and distinct variety.

1. Medium vigor;

2. Semi-erect growth habit;

3. Medium size, pentagonal to circular shaped leaves with medium green upper surfaces and pale green lower surfaces and medium texture;

4. Short-medium size, dense, cylindrical shaped berry clusters with one middle-size wing; dark blue berries with soft to slightly firm flesh, herbaceous taste, and no flesh coloration; primarily used for wine; maturing in Mid-late September (Middle Friuli, North-Eastern Italy);

5. Primocanes and floricanes with elliptic cross section, brownish color, no lenticels, and short-middle internodes (13-14 cm with diameter: 9-10 mm);

6. Harvesting time of Mid-late September (Middle Friuli, North-Eastern Italy); and

7. Resistance to winter temperature (−24° C.), resistance to downy mildew, and tolerance to powdery mildew.

Plants of the new grapevine ‘ROYAL CABERNET’ differ from plants of the parents, Vitis vinifera ‘Cabernet Sauvignon’ (unpatented) and Vitis cross ‘20/3’ (unpatented), in the following characteristics described in Table 1.

TABLE 1
Comparison with parent varities

| | | Female Parent | |
|---|---|---|---|
| | New Cultivar | ‘Cabernet | Male Parent |
| Charac- | ‘ROYAL | Sauvignon’ | ‘20/3’ |
| teristic | CABERNET’ | (unpatented) | (unpatented) |
| vigour: | medium | strong | medium |
| growth | semi-erect | horizontal | semi-erect |
| habit | | | |
| leaf | medium size, | medium size, | Medium size, |
| | medium green | medium green | light green col- |
| | color (upper | color (both up- | or (upper sur- |
| | surface), pale | per and lower | face), green |
| | green color (low- | surface), low | color (lower |
| | er surface), pen- | hair density in | surface), no hairs |
| | tagonal and cir- | both surfaces, | in both surfaces, |
| | cular shape, med- | circular shape, | wedge-shaped- |
| | ium texture, no | medium texture | kidney-shaped, |
| | hairs in both sur- | | medium texture |
| | faces. | | |
| cluster | short-medium | Very low weight, | low weight, con- |
| | size cluster, cylin- | cylindrical shape | ical, with 1-2 |
| | drical with one | with 1-2 wings, | wings, medium |
| | middle-size wing, | medium bunch | dense, berry skin |
| | dense, berry skin | density, berry | with green-yellow |
| | with dark-blu col- | skin with blue | color, slightly firm |
| | or, soft to slightly | black color, soft | flesh, neutral taste, |
| | firm flesh, herba- | flesh, neutral | no flesh coloration |
| | ceous taste, no | taste, no to very | |
| | flesh coloration | weak flesh col- | |
| | | oration | |
| primo- | ellictic cross sec- | circular cross | oblate cross sec- |
| cane and | tion, brownish | section, brown- | tion, brownish |
| floricane | colour, lenticels: | ish color, lenti- | color, lenticels: ab- |
| | absent, short-mid- | cels: absent, med- | sent, medium inter- |
| | dle internodes | ium internodes, | nodes, diameter |
| | (13-14 cm), diam- | diameter small | small (about 8 mm) |
| | eter: 8 9-10 mm | (about 8 mm) | |
| harvest- | Mid-late Septem- | late (1st week of | Early (last decade |
| ing time | ber (Middle Friuli, | October in Mid- | August, Middle |
| | North-Eastern | dle Friuli, North- | Friuli, North- |
| | Italy) | Eastern Italy) | Eastern Italy) |
| resis- | resistant to win- | Resistance to win- | resistant to winter |
| tances | ter temperature | ter temperature not | temperature not |
| | (−24° C.), resis- | checked, suscepti- | checked, resistant |
| | tant to downy | ble to downy mil- | to downy mildew, |
| | mildew, tolerant | dew, susceptible | resistant to pow- |
| | to powdery mil- | to powdery mil- | dery mildew |
| | dew. | dew | |

Of the many commercial cultivars known to the present inventor, the most similar to the new grapevine ‘ROYAL CABERNET’ is the female parent ‘Cabernet Sauvignon’, to which a comparison has been provided above.

### BRIEF DESCRIPTION OF THE PHOTOGRAPHS

The accompanying photographs illustrate the overall appearance of the new grapevine ‘ROYAL CABERNET’ showing the colors as true as is reasonably possible with colored reproductions of this type. Colors in the photographs may differ slightly from the color values cited in the detailed botanical description, which accurately describe the color of ‘ROYAL CABERNET’.

FIG. 1A and FIG. 1B show a typical fruit cluster of ‘ROYAL CABERNET’ taken on Sep. 10, 2012, in Udine, Italy

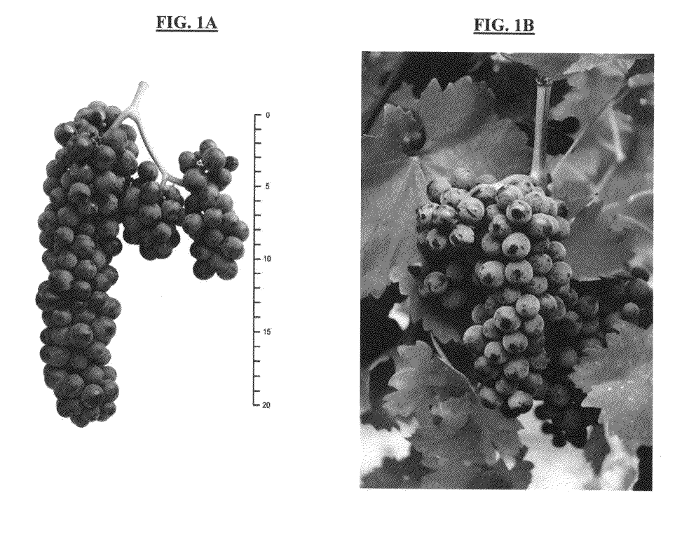

FIG. 2 shows a typical mature leaf (upper surface, left and lower surface, right) of ‘ROYAL CABERNET’ taken on Jun. 15, 2012, in Udine, Italy.

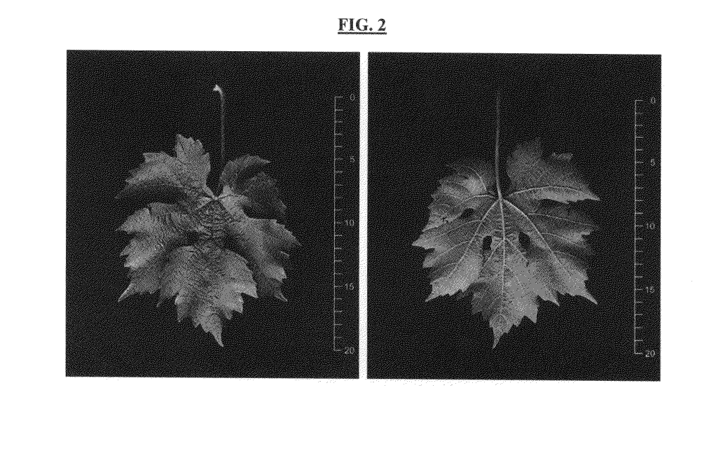

FIG. 3 shows a typical mature vine of ‘ROYAL CABERNET’ taken on Aug. 3, 2012.

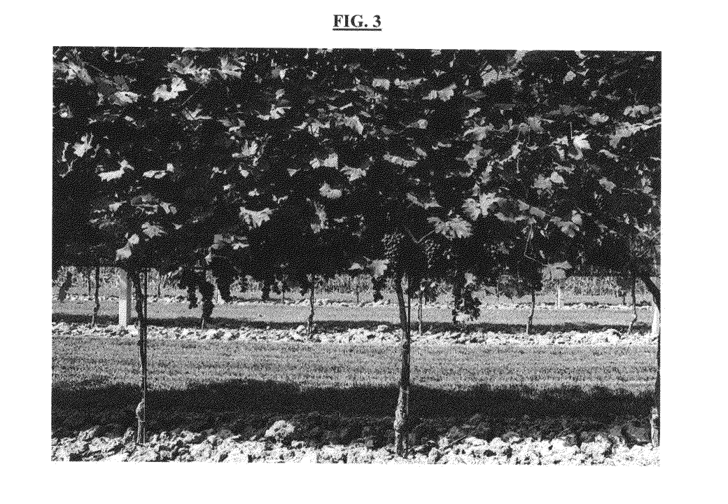

FIG. 4 shows a close-up view of mature fruit of ‘ROYAL CABERNET taken on Sep. 10, 2012, in Udine, Italy.

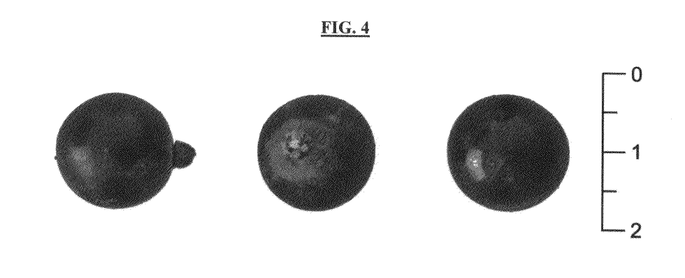

### DETAILED BOTANICAL DESCRIPTION

The following description of ‘ROYAL CABERNET’ unless otherwise noted, is based on observations taken during the 2011, 2012, and 2013 growing seasons(s) in Udine, Italy and Fossalon di Grado (GO), Italy. from plants dug from a nursery located in Rauscedo (PN), Italy during the beginning of December 2007 and planted approximately 16 to 20 weeks later in Udine, Italy and Fossalon di Grado (GO), Italy The phenotypical descriptions and color designations stated for the new variety may vary, depending upon variations in environmental factors, including weather (temperature, humidity and light intensity), day length, soil type, location and cultural conditions. ‘ROYAL CABERNET’ has not been observed under all possible environmental conditions.

| | | |
|---|---|---|
| | | ‘CABERNET |
| | ‘ROYAL | SAUVIGNON’ |
| Characteristic | CABERNET’ | (unpatented) |

GENERAL

| | | |
|---|---|---|
| Resistance to | Resistant to downy mildew, | Susceptible to both downy |
| pest/disease | tolerant to powdery mildew | and to powdery mildew |
| Resistance to | Resistant to winter cold | Resistant to winter cold |
| natural elements | down to −23° C. | not evaluated |

VINE

| | | |
|---|---|---|
| Vigor | medium | high |
| Trunk diameter | 6 cm (6 yrs-old plant) | 6 cm (6 yrs-old plant) |
| Bark | | |
| color | Dark brown | Brownish |
| underbark color | Light brown | Light brown |
| texture | n.a. | n.a. |
| Canes | | |
| length | Internode length | Internode length |
| | cm 13-14 | cm 10 |
| width | 9-10 mm | 8 mm |
| Shoots | | |
| shape | Semi-erect, cross section: | Horizontal, cross section: |
| | elliptic, surface: with | circular, surface: smooth, |
| | stripes, nodes and inter- | nodes and internodes |
| | nodes without hairs | without hairs |
| color | Internodes with red and | internodes and nodes with |
| | green pigmentation on | green and red color on |
| | dorsal side, and green | dorsal side and green pig- |
| | ventral side | mentation on ventral side |
| Tendrils | | |
| form | Bifid | bifid |
| color | Green | Green |
| texture | normal | normal |
| number | 2 or <2 consecutive | 2 or less consecutive |
| Buds | | |
| size | average | average |
| shape | round | round |
| color | Light brown | Light brown |
| number | 2/node | 2/node |
| time of | early | medium |
| budbreak | | |

LEAVES

| | | |
|---|---|---|
| Size | medium | medium |
| Number of | 5 | 5 |
| leaflets | | |
| Glossiness | Medium-high | medium |
| Cross section | V-shaped | flat |
| shape | | |
| Color (immature) | | |
| Upper surface | pale green | copper-reddish |
| Under surface | pale green | copper-reddish |
| Color (mature) | | |
| Upper surface | Medium green | medium green |
| Under surface | pale green | medium green |
| Petiole | | |
| Length (cm) | 6.2 | 6.0 |
| Color (upper | green | reddish |
| surface) | | |
| Color (under | Sometimes reddish | reddish |
| surface) | | |
| Stipule | n.a. | n.a. |
| orientation | | |

FLOWERS

| | | |
|---|---|---|
| Flowering | End of May | 1st week June |
| period (time of | | |
| beginning of | | |
| flowering) | | |
| Sex | hermaphrodite | hermaphrodite |
| Size | Average | average |
| Diameter (cm) | 0.3 (stamens) | 0.3 (stamens) |
| Fragrance | average | average |
| Flower number | n.a. | n.a. |
| (at 3^{rd} node | | |
| from tip of | | |
| lateral mean | | |
| and range) | | |
| Petals | | |
| Length (cm) | n.a. | n.a. |
| Width (cm) | n.a. | n.a. |
| Overall shape | calyptra | calyptra |
| Color (immature) | green | green |
| Upper surface | | |
| Under surface | | |
| Color (mature) | green | green |
| Upper surface | | |
| Under surface | | |
| Sepals | | |
| Length (cm) | n.a. | n.a. |
| Width (cm) | n.a. | n.a. |
| Overall shape | n.a. | n.a. |
| Color (immature) | n.a. | n.a. |
| Upper surface | | |
| Under surface | | |
| Color (mature) | n.a. | n.a. |
| Upper surface | | |
| Under surface | | |
| Pedicel | | |
| Length (mm) | 45 (cluster) | 6 |
| Color | green | green |

FRUIT

| | | |
|---|---|---|
| Primocane time | September 10th | September 27th |
| of fruiting (1^{st} | | |
| pick) | | |
| Clusters | | |
| cluster weight | g 130 | Small (g 139) |
| cluster shape | cylindrical | cylindrical |
| cluster length | mm 180 | medium (mm 195) |
| avg. berries per | 120 | 99 |
| cluster | | |
| avg. clusters per | 2 | 2 |
| shoot | | |
| Berries | | |
| Berry size | g 1.44 | Very low (g 1.4) |
| Berry length | 1.76 | 1.5 |
| (cm) | | |
| Berry width | 1.56 | 1.5 |
| (cm) | | |
| Overall shape | globose | globose |
| of berry | | |
| Berry Texture | soft to slightly firm | soft |
| Berry Color | green | green |
| (immature) | | |
| Berry Color | Dark-blu | Blu black |
| (mature) | | |
| Soluble solids | 24.8 | 22.1 |
| (%) | | |
| Titratable | 4.8 | 7.4 |
| acidity (as g/L | | |
| tartaric acid) | | |
| Sugar/acid ratio | 5.2 | 3.0 |
| Firmness | soft | soft |
| Seeds | 2-3 | 2-3 |
| Skin cracking? | no | no |
| Juice color | red | No or very weak |
| Berry taste | Slightly herbaceous | herbaceous |
| Eating quality | n.a. | n.a. |
| Berry uses | wine | wine |
| Shipping | n.a. | n.a. |
| quality | | |

What is claimed is:

1. A new and distinct grapevine, referred to as ‘ROYAL CABERNET, as herein described and illustrated by the characteristics set forth above.
